(19) United States
(12) Patent Application Publication
(10) Pub. No.: US 2021/0100991 A1
(43) Pub. Date: Apr. 8, 2021

(54) METAL OXIDE AND POLYMER-CONTROLLED DELIVERY SYSTEMS, SUNSCREENS, TREATMENTS, AND TOPICAL COATING APPLICATORS FOR ANTIFUNGAL AND ANTIMICROBIAL AGENTS

(71) Applicant: Biointraface Inc., Coventry, RI (US)

(72) Inventor: John D. Jarrell, East Greenwich, RI (US)

(21) Appl. No.: 17/024,472

(22) Filed: Sep. 17, 2020

Related U.S. Application Data
(60) Provisional application No. 62/901,602, filed on Sep. 17, 2019.

(51) Int. Cl. A61M 35/00; A61J 1/06; A61J 1/20; A61M 25/00; A61F 2/28; A61B 17/84; A61B 17/86; A61B 17/80; A61B 17/60; A61F 2/30; A61F 13/00; A61M 16/04; A61B 46/00; A61K 47/02; A61K 31/47; A61K 31/085; A61K 31/4164; A61K 31/79; A61K 31/325; A61K 31/201; A61K 38/12; A61K 31/65; A61K 31/702; A61K 31/327; A61K 31/05; A61K 31/60; A61K 33/04; A61K 6/69; A61L 29/10; A61L 31/10; A61L 15/22; A61L 27/34; A61L 27/54

(52) U.S. Cl. CPC A61M 35/003; A61F 2310/00389; A61J 1/2093; A61M 25/0045; A61F 2/28; A61B 17/846; A61B 17/866; A61B 17/80; A61B 17/60; A61F 2/30; A61F 13/00063; A61M 16/0402; A61B 46/40; A61K 47/02; A61K 31/47; A61K 31/085; A61K 31/4164; A61K 31/79; A61K 31/325; A61K 31/201; A61K 38/12; A61K 31/65; A61K 31/702; A61K 31/327; A61K 31/05; A61K 31/60; A61K 33/04; A61K 6/69; A61L 29/106; A61L 31/10; A61L 15/22; A61L 27/34; A61L 27/54; A61J 1/06

(57) ABSTRACT

A dose applicator for preparing a mixture at a point of use includes a housing defining an interior and an opening. First and second ampoules are contained in the interior, wherein the first ampoule contains a first component and the second ampoule contains a second component comprising one or more antifungal agent. An applicator tip is secured in the opening so that when the first and second ampoules are broken, the first and second components mix to form the mixture and the mixture can egress from the application tip.

### FIELD OF THE INVENTION

The embodiments of the present invention relate to methods and systems for mixing and applying two or more components at the point of use, and more particularly to improved applicators for mixing and applying various ingredients of a coating. The embodiments of the present invention further relate to compositions, as well as related methods, coatings, and delivery mechanisms for antifungal and antimicrobial drugs.

### BACKGROUND OF THE INVENTION

The greatest obstacle for transdermal drug delivery is the stratum corneum that forms a primary rate limiting barrier to the permeation of drugs across the skin. It consists of dead, flattened cells filled with keratin that are embedded in a lipid matrix. The stratum corneum has been described as hydrophilic protein bricks embedded in a hydrophobic lipid mortar. There has been a considerable interest in the potential usefulness of the topical application of non-steroidal anti-inflammatory drugs (NSAIDs) such as ibuprofen, indomethacin and diclofenac. These weak acidic drugs are effective in the treatment of rheumatoid arthritis and osteoarthritis.

However, the disadvantage of the topical route for drug delivery is that a relatively high dose is required to deliver therapeutic amounts of drug across the skin. To improve the topical delivery of drugs, several strategies are available including the use of penetration enhancers and optimization of drug release from the formulation. The pH of the formulation has an impact on the penetration rate of weak acidic and weak basic drugs.

The flux of ibuprofen from a saturated solution at pH values ranging from 2.2 to 9.0 using human skin in vitro has been studied. It was reported that flux of the drug increased with an increase in the pH of the solution. The reason behind this effect was unclear. However, ibuprofen was reported to show a considerable surface activity. Since surfactants are well known penetration enhancers, it is possible that ibuprofen has acted as surfactant and thus impaired the skin permeability barrier. Hence, it is planned to study effect of ibuprofen concentration, in saturated solutions, on its permeation across skin. It is a known concept that an increase in concentration of drug in the vehicle results in enhanced flux due to increased thermodynamic activity. One delivery mechanism was designed by keeping thermodynamic activity constant while increasing the drug concentration in the delivery mechanism. This was done by preparing saturated solutions of ibuprofen, of different concentrations, using disodium hydrogen phosphate solutions of various molar strengths. The permeation of ibuprofen from its saturated solutions across rat epidermis and human epidermis was studied, and the results were compared with those obtained from silastic membrane.

The transdermal route now ranks with oral treatment as the most successful innovative research area in drug delivery, with around 40% of the drug delivery candidate products under clinical evaluation related to transdermal or dermal system. The worldwide transdermal patch market approaches £ 2 billion, based on only ten drugs including scopolamine, nitroglycerine, clonidine, estrogen, testosterone, fentanyl, and nicotine, with a lidocaine patch soon to be marketed.

Antifungal drugs also suffer from poor transdermal delivery. There are numerous side effects associated with oral delivery of antifungal drugs, including, nausea, abdominal pain, diarrhea, headache, rash, allergic reaction (swelling of your face, neck or tongue or difficulty breathing), severe skin reaction (such as peeling or blistering skin), and liver damage.

Accordingly, there is a need for safer, more effective delivery mechanisms for antifungal drugs.

### BRIEF SUMMARY OF THE INVENTION

The embodiments of the present invention provide provides safer, more effective methods, coatings, and delivery mechanisms for antifungal and antimicrobial drugs.

The success of a dermatological drug to be used for systemic drug delivery depends on the ability of the drug to penetrate through skin in sufficient quantities to achieve the desired therapeutic effect. The subject technology uses chemical penetration enhancers and the associated possible mechanisms of action. Reservoir and barrier systems are commonly used for controlled release in applications such as transdermal drug delivery and with implantable systems of similar design. The drug or active ingredient is often in a liquid or gel state and is delivered across a rate controlling polymer membrane. These systems have the advantage of providing near zero order release characteristics. This means that such a system delivers a consistent amount of drug or active ingredient over an extended period of time. One disadvantage of such systems is the sudden and uncontrolled release of drug or active ingredient if the barrier is disrupted or a defect is present in the barrier or rate controlling membrane. To reduce the occurrence of leakage resulting in the uncontrolled release of drugs or active ingredients, some delivery systems use a solid matrix delivery system. This consists of a polymer or mixture of copolymers, drugs or active ingredients with or without a rate controlling membrane and often agents designed to improve release and skin permeation properties.

Metal oxide based delivery systems have been described in a past patent¹ and published in the literature.² In these cases, the drugs or active ingredients are distributed within a matrix of metal oxides and applied in the form of coatings or bulk materials. One drawback of these systems is that it is difficult to deliver much of the drug or active ingredient loaded into the matrix. These dopants become trapped within the matrix.

In the case where refractory and transitional metal oxide matrices consisting of titanium, zirconium, niobium and or tantalum oxides are doped with water soluble metals or metal oxides of silver, vanadium, zinc and or copper and similar agents to provide bioactive and antimicrobial activity, much of the doped ingredients become trapped within the matrix. This was demonstrated in a publication by Jarrell et al.³ In these experiments, coatings of pure titanium oxide matrices doped with vanadium oxide, released vanadium at a much lower rate than similarly doped titanium oxide and polymer hybridized matrix coatings.

In a liquid formula, a barrier includes a polymer or metal oxide. In an antifungal formula, the liquid compositions are precursors, where metal oxide can be added (not using two layers). For antifungal drug delivery, a layer makes a reservoir coating—made from silver plus a polymer—with or without diffusion layers. It can be two separate layers, either layer or both of a reservoir and barrier layer. There can also be two applicators with one or more layers in each applicator, either of which can form film of the barrier coating.

The success of a dermatological drug to be used for systemic drug delivery depends on the ability of the drug to penetrate through skin in sufficient quantities to achieve the desired therapeutic effect. The subject technology uses chemical penetration enhancers and the associated possible mechanisms of action.

Reservoir and barrier systems are commonly used for controlled release in applications such as transdermal drug delivery and with implantable systems of similar design. The drug or active ingredient is often in a liquid or gel state and is delivered across a rate controlling polymer membrane. These systems have the advantage of providing near zero order release characteristics. This means that such a system delivers a consistent amount of drug or active ingredient over an extended period of time. One disadvantage of such systems is the sudden and uncontrolled release of drug or active ingredient if the barrier is disrupted or a defect is present in the barrier or rate controlling membrane. To reduce the occurrence of leakage resulting in the uncontrolled release of drugs or active ingredients, some delivery systems use a solid matrix delivery system. This consists of a polymer or mixture of copolymers, drugs or active ingredients with or without a rate controlling membrane and often agents designed to improve release and skin permeation properties.

Metal oxide based delivery systems have been described in a past patent patents (U.S. Pat. No. 7,906,132, Anti-infectious, biocompatible titanium coating for implants, and method for the production thereof to Ziegler et al. issued on Mar. 15, 2011) and published in the literature. (Jarrell J D, Dolly B, Morgan J R. Controlled release of vanadium from titanium oxide coatings for improved integration of soft tissue implants. J Biomed Mater Res A, Volume 90A, Issue 1, Pages 272-281, June 2009). In these cases, the drugs or active ingredients are distributed within a matrix of metal oxides and applied in the form of coatings or bulk materials. One drawback of these systems is that it is difficult to deliver much of the drug or active ingredient loaded into the matrix. These dopants become trapped within the matrix.

In the case where refractory and transitional metal oxide matrices consisting of titanium, zirconium, niobium and or tantalum oxides are doped with water soluble metals or metal oxides of silver, vanadium, zinc and or copper and similar agents to provide bioactive and antimicrobial activity, much of the doped ingredients become trapped within the matrix. This was demonstrated in a publication by Jarrell et al. (Jarrell J D, Dolly B, Morgan J R. Rapid screening, in vitro study of metal oxide and polymer hybrids as delivery coatings for improved soft-tissue integration of implants. J Biomed Mater Res A, Volume 92A, Issue 3, Pages 1094-1104, 1 Mar. 2010). In these experiments coatings of pure titanium oxide matrices doped with vanadium oxide, released vanadium at a much lower rate than similarly doped titanium oxide and polymer hybridized matrix coatings.

In a liquid formula, a bather includes a polymer or metal oxide. In a suntan lotion formula, the liquid compositions are sunscreen precursors, where metal oxide can be added (not using two layers). For drug delivery, a layer makes a reservoir coating—made from silver plus a polymer—with or without diffusion layers. It can be two separate layers, either layer or both of a reservoir and barrier layer. There can also be two applicators two applicators with one or more layers in each applicator, either. Either of which can form film of the bather coating.

It should be appreciated that the subject technology can be implemented and utilized in numerous ways, including without limitation as a process, an apparatus, a system, a device, a method for applications now known and later developed. These and other unique features of the system disclosed herein will become more readily apparent from the following description and the accompanying drawings.

### BRIEF DESCRIPTION OF THE DRAWINGS

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1A illustrates a plot (a) in accordance with the subject technology.

FIG. 1B illustrates a plot (b) in accordance with the subject technology.

FIG. 1C illustrates a plot (c) in accordance with the subject technology.

FIG. 1D illustrates a plot (d) in accordance with the subject technology.

FIG. 2 is a cross-sectional view of a coated article in accordance with the subject technology.

FIG. 3 is a cross-sectional perspective view of a coated catheter in accordance with the subject technology.

FIG. 4 illustrates a sustained release delivery system in accordance with the subject technology.

FIG. 5 illustrates a view of another sustained release delivery system in accordance with the subject technology.

FIG. 6A shows a tandem pop ampoule dose applicator in accordance with the subject disclosure.

FIG. 6B shows a parallel pop ampoule dose applicator in accordance with the subject disclosure.

### DETAILED DESCRIPTION OF THE INVENTION

It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the term antimicrobial represents an agent that kills microorganisms, and/or inhibits or stops their growth. The major groups of microbes are namely bacteria, archaea, fungi (yeasts and molds), algae, protozoa, viruses. For the purpose of this description prions are included as microbes and antiprion agents as a subtype of antimicrobial agents. Antimicrobials can be grouped according to the microorganisms they act primarily against. An antibiotic is a type of antimicrobial substance active against bacteria. It is a type of antibacterial agent for fighting bacterial infections, and antibiotic medications are widely used in the treatment and prevention of such infections. They may either kill or inhibit the growth of bacteria. A limited number of antibiotics also possess antiprotozoal activity. Antibiotics may not be effective against viruses such as the common cold or influenza. Antimicrobial agents which inhibit viruses are termed antiviral drugs or antivirals.

As used herein, the terms “effective amount” and “therapeutically-effective amount” include an amount sufficient to prevent or ameliorate a manifestation of disease or medical condition, such as fungal infection. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treating” or “treatment” or “to treat” or “alleviating” or “to alleviate” refer to both (1) therapeutic measures that cure, slow down, lessen symptoms of, and/or halt progression of a diagnosed disease or infection and (2) prophylactic or preventative measures that prevent or slow the development of a disease or infection.

As used herein, the term “long-term” administration means that the therapeutic agent or drug is administered for a period of at least 30 days. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more. A “short term” administration means that a therapeutic agent or drug is administered over a period of 24 hours. A “medium term” administration means that a therapeutic agent or drug is administered for a period more than 24 hours and less than 30 days.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;⁴ The Encyclopedia of Molecular Cell Biology and Molecular Medicine;⁵ Molecular Biology and Biotechnology: a Comprehensive Desk Reference;⁶ Immunology;⁷ Janeway's Immunobiology;⁸ Lewin's Genes XI;⁹ Molecular Cloning: A Laboratory Manual;¹⁰ Basic Methods in Molecular Biology;¹¹ Laboratory Methods in Enzymology;¹² Current Protocols in Molecular Biology (CPMB);¹³ Current Protocols in Protein Science (CPPS);¹⁴ and Current Protocols in Immunology (CPI).¹⁵

Other terms are defined herein within the description of the various aspects of the invention.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “solvent” is intended to include polar and non-polar solvents. The term polar solvent as used herein includes solvents that have a large dipole moment and have bonds between atoms with different electronegativities. Examples of such atoms include oxygen and hydrogen, nitrogen and hydrogen. Polar solvents can be protic and contain a labile H⁺ and such solvent molecules donate protons (Hi) to solutes generally via hydrogen bonding. Water is one example of a protic solvent. Other illustrative examples of protic solvents are n-butanol, ethanol, propanol, acetic acid, and methanol. Polar solvents also include aprotic solvents which cannot donate hydrogen. Illustrative examples of aprotic polar solvents include acetone, dimethyl sulfoxide, dichloromethane, ethyl acetate, acetonitrile, THF, and DMF (N,N-dimethylformamide).

The term non-polar solvent as used herein includes solvents that contain bonds between atoms with similar electronegativities, such as carbon and hydrogen. Bonds between atoms with similar electronegativities lack partial charges and this absence of charge makes these molecules non-polar. Non-polar solvents generally have low dielectric constants (<5) and are not good solvents for charged species such as anions. Illustrative examples of non-polar solvents include hexane, pentane, benzene, toluene, dioxane, chloroform, and diethyl ether. The term non-polar solvent also includes neutral solvents as understood by one skilled in the art.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

For use in the methods of this invention, active compounds can be given per se or as a pharmaceutical composition containing, for example, 0.1 to 99.5% (more preferably, 0.5 to 90%) of active ingredient in combination with a pharmaceutically-acceptable carrier.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art.¹⁶

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

### Metal Oxide and Polymer-Controlled Delivery Systems

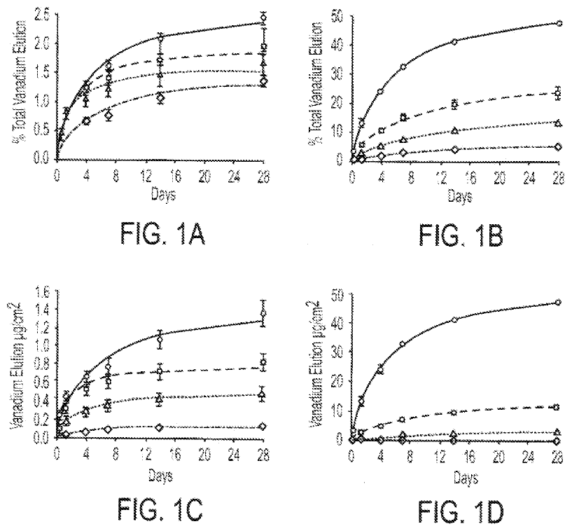

Referring to FIG. 1, four plots (FIG. 1A-D) are shown. The top plots (FIG. 1A-B) illustrate how addition of Polydimethylsiloxane (PDMS) to titanium coatings increases the elution rate of vanadium. FIG. 1A-B present the percentage of total vanadium loading released into PBS as a function of time for titanium oxide coatings without PDMS (left, open symbols) and 66.6% Titanium oxide—33.4% PDMS hybrid coatings (right, closed symbols), while the lower plots (FIG. 1C-D) are the cumulative release rates per surface area over the same period for four different vanadium concentrations, 20% (circles), 10% (squares), 5% (triangles) and 1.25% (diamonds).

Modeling curves of vanadium release are shown for 20% ( [symbol] ) 10% ( [symbol] ), 5% ( [symbol] ) and 1.25% ( [symbol] ) vanadium doping. As can be seen, titanium oxide doped with 20% vanadium oxide released approximately 2.5% of the total vanadium loading after 28 days. However, 66.6% Titanium oxide and 33.4% Polydimethylsiloxane hybrid coatings doped with the same amount of vanadium oxide (20%), released approximately 50% of the total vanadium loading after 28 days. The result being that polymer hybridization of the metal oxides improved the rate of delivery of dopants.

One factor which limits the use of coatings as a delivery matrix is the total volume available for drug loading. The limit is constrained by the thickness and area of the coatings. This is compounded by the fact that the metal oxides and polymers selected for the matrix (i.e., titanium oxide, tantalum oxide, zirconium oxide, niobium oxide) are generally used to both improve the biocompatibility of the surface coating with surrounding tissues and cells and control the rate of delivery of the doped agent. If excessive levels of doping are used in the matrix (for example, over 50% Ag or V) then the properties of the matrix materials are increasingly lost, delivery rates may become excessively high and outside of the therapeutic range and sustained release can be compromised and prevented.

To overcome these problems and further enhance and prolong the delivery from metal oxide and polymer hybrid coating systems, multiple layers of coatings may be used. These can be multiple layers of the same coatings. However, there is a limitation to number and thickness of coatings that can be used without compromising the mechanical properties and aesthetic appearance of the coatings.

To overcome the limitations of multiple coatings of the same composition, the subject technology includes a multi-layer coating system, where the innermost layer or layers contain a higher concentration of the active ingredient or ingredients and act as a reservoir, while the outer layer or layers contain lower concentrations of the active ingredient or ingredients and act as a rate controlling diffusion barrier.

When exposed to liquids or bodily fluids, the liquid diffuses through the barrier coating and releases the active ingredient into the surrounding liquids or bodily tissues. The active ingredients in the reservoir layer move down the diffusion gradient and replace the ions or active ingredients lost within the barrier layer, thus provided sustained and prolonged release over the single or multiple coatings without the reservoir layer design. This way the biocompatible and rate limiting properties of the barrier layer or layers are also maintained. Put in other terms, the outermost coating acts as a diffusion barrier, while the underlying coatings can function as solid state, gel, sol or xerogel reservoir containing higher concentrations of the active agents for elution or controlled delivery through the rate controlling diffusion barrier. This is an improvement upon existing systems using single or multiple coatings containing the same or similar levels of the active ingredient for elution. This has the advantage of providing a reservoir for allowing for longer, sustained release through the coatings systems. The use of a rate controlling barrier film also prevents excessively high release of the active ingredient when such can have negative therapeutic results. This allows the diffusion barrier to have lower levels of delivered agents and be optimized for tissue contact, cellular adhesion, cell proliferation, inflammatory properties and biocompatibility, since it is no longer the primary reservoir for the delivered agent.

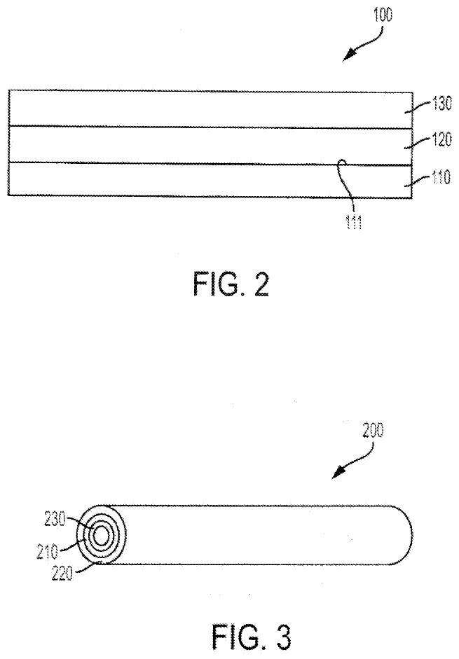

Referring now to FIG. 2, a cross-sectional view of a coated article 100 is shown. The coated article 100 includes an object 110 and a reservoir coating 120 supported on a surface 111 of object 110 and a diffusion barrier coating 130. In general, upper coating 130 includes a metal oxide and a polymer, while lower coating 120 includes a higher level of active ingredients for delivery with or without additional matrix forming metal oxides and polymers. The metals and metal oxides that can be used in coating 120 generally include those which exhibit one or more therapeutic effects under a certain condition. In some embodiments, the metal oxide in coating 120 is a transition metal oxide. Examples of suitable metal oxides for use in coating 120 include titanium oxides, vanadium oxides, zinc oxides, zirconium oxides, silver oxides, tantalum oxides, or combinations thereof. In other embodiments coating 120 consists of a drug, biologic, peptide, protein, glycoprotein, polysaccharide, phage, virus, prion, bacteria, DNA, RNA, gene. In some embodiments, the metal oxide in coating 130 is a transition metal oxide, titanium oxide, tantalum oxide, zirconium oxide, zinc oxide, niobium oxide. In other embodiments, coating 130 consists of a metal oxide and a polymer or copolymer.

The polymer or copolymer used in one or all of the coating layers may be functionalized, such as a siloxane with methoxy and amine groups. In such a case, the functional groups may be used to tether or immobilize drugs, antibiotics, biologics, proteins, peptides, phages, and other molecules traditionally stabilized to surfaces using layer by layer self-assembly and silanization techniques. The advantage of type of the described wet chemistry, sol-gel type hybridization over the prior art is that the same coating can function as a non-resorbable bioactive surface, a diffusion barrier and a delivery matrix while having other active agents immobilized to the surface of the barrier layer. In other cases, the polymer or copolymer can be based on polyurethanes.

An example of a medical device having multiple coatings described above is a coated catheter. As shown in FIG. 3, a catheter 200 includes a catheter body 230, a coating reservoir 210 supported by the outer surface of catheter body 230, and a barrier coating 220 supported by the inner surface of reservoir coating 210. In some embodiments, both coatings 210, 220 can include a metal oxide (e.g., a titanium oxide) and a polymer. In some embodiments, coatings 210, 230 can have different compositions (e.g., different metal oxides and/or different polymers).

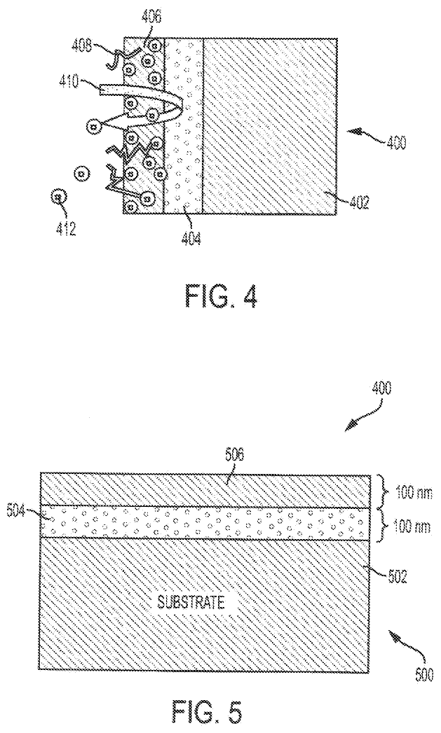

A more detailed schematic of a delivery system 400 is shown in FIG. 4 and FIG. 5. The delivery system 400 shows a general and specific example of a reservoir and barrier system in FIG. 4 and FIG. 5, respectively. The delivery system 400 may be applied to catheters, dental implants, provisional dental restorations, orthopaedic trauma implants, nails, bone screws, bone plates, external fixation pins and wires, joint replacements, and wound dressings as well as urinary catheters, blood contacting catheters, endotracheal tubes and other airway management devices. The delivery system 400 may be applied to hospital furniture, disposable cloths and drapes, medical and surgical instruments and equipment to prevent bacteria growth and infection through sustained delivery of silver ions, copper ions, zinc, biologics or drugs. The system 400 may be applied directly to the skin or epithelial layers for transepithelial pharmacological delivery or applied to internal organs of the body for localized and systemic delivery of bioactive agents.

A composition used to form a metal oxide and polymer coating, methods of preparation and application are disclosed in U.S. Pat. No. 10,668,259, which create a long lasting sunscreen. After application of the composition, the metal oxide precursor decomposes and polymerizes with the polymer precursor to form a coating of metal oxide and polymer which also binds to the surface of the skin, providing long lasting water resistance and protection from UVA and UVB. The metal oxide consists of a UV absorbing mineral such as titanium oxide, zinc oxide or a mixture of the two. The polymer consists of at least one functionalized polymer which binds to keratin and facilitates copolymerization of the metal oxide and polymer precursors. An example is provided in in U.S. Pat. No. 10,668,259. Example 8 included the use of the metal oxide and polymer forming solutions in conjunction with FDA recognized sunscreens, to form a sunscreen that is water resistant. The added ingredients include: Aminobenzoic acid (PABA) up to 15 percent; Avobenzone up to 3 percent; Cinoxate up to 3 percent; Dioxybenzone up to 3 percent; Homosalate up to 15 percent; Menthyl anthranilate up to 5 percent; Octocrylene up to 10 percent; Octyl methoxycinnamate up to 7.5 percent; Octyl salicylate up to 5 percent; Oxybenzone up to 6 percent; Padimate O up to 8 percent; Phenylbenzimidazole sulfonic acid up to 4 percent; Sulisobenzone up to 10 percent; Titanium dioxide up to 25 percent; Trolamine salicylate up to 12 percent; Zinc oxide up to 25 percent; Ensulizole up to 4 percent; Homosalate up to 15 percent; Meradimate up to 5 percent; Octinoxate up to 7.5 percent; Octisalate up to 5 percent; Octocrylene up to 10 percent; Oxybenzone up to 6 percent; and Padimate O up to 8 percent.

The subject technology also includes systems for delivery of the coatings to the point of use. The systems include dose applicator swabs and spray applicators. One form of this system is a single-use pop ampoule applicator, which contains one, two, three or more glass or plastic ampoules. The applicator system and contents are either sterile or non-sterile depending on the end use. The ampoules also contain the solvents used to suspend the precursors, like alcohols (including monohydric alcohols, polyhydric alcohols, unsaturated aliphatic alcohols, alicyclic alcohols and their mixtures as suitable solvents and carriers), isopropanol, butanol, ethanol, methanol, and heptanes, hexanes, xylenes, terpenes, terpineol, lavender oi, (R)-(+)-LIMONENE, (S)-(−)-LIMONENE, ALPHA-TERPINENE, Orange Terpinene. Tandem pop ampoule dose applicator. Metal oxide and polymer precursors and active agents like silver compounds mixed with solvents such as isopropanol, butanol, hexanes, xylene, heptanes in ampoules. Crushing of the ampoules releases the contents and allows the components to mix and flow out to the applicator tip. The coating is then brushed or dabbed onto the desired surface.

Referring in particular to FIG. 4, a sustained release hybrid delivery system 400 is shown in cross-section. The system 400 can be application to an item 402 such as a trauma nail, pin, screw, endoprosthesis, catheter and the like. The item 402 can be any kind of substrate such as Titanium, Cobalt-Chromium, stainless steel type 316, polymers and the like.

A reservoir layer 404 is applied to the substrate 402. The reservoir layer 404 can be concentrated bioactive and anti-microbial agents such as ion forming metals like Silver, Copper, Zinc, Vanadium. The reservoir layer 404 can also include drugs, bioactive molecules and/or reagents like iron compounds. A bioactive diffusion layer 406 is applied to the reservoir layer 404. The bioactive diffusion layer 406 can include immobilized drugs or crystalline titanium oxide 408. Preferably, water can diffuse into the coatings 408 as represented by arrow 410. When an item 402 is coated, sustained delivery out to adjacent tissues is accomplished as indicated by items 412. The items 412 may be metal ions like silver, copper, zinc and vanadium, drugs, bioactive molecules, and reaction agents like iron.

Referring again to FIG. 5, another cross-sectional view of a system 500 is shown. The system 500 has a similar structure to system 400 and thus similar portions are references as a “5” series number instead of a “4” series number. The reservoir layer 504 and the bioactive diffusion layer 506 can both be about 100 nm thick. In one example, the reservoir layer is (Ag₂O)_{y}(TiO₂)(CₐH_{b}OSi)_{z}. The bioactive diffusion layer 506 has a rate controlling coating of (TiO₂)ₓ(Ag₂O)_{y}(TiO₂)(CₐH_{b}OSi)_{z}.

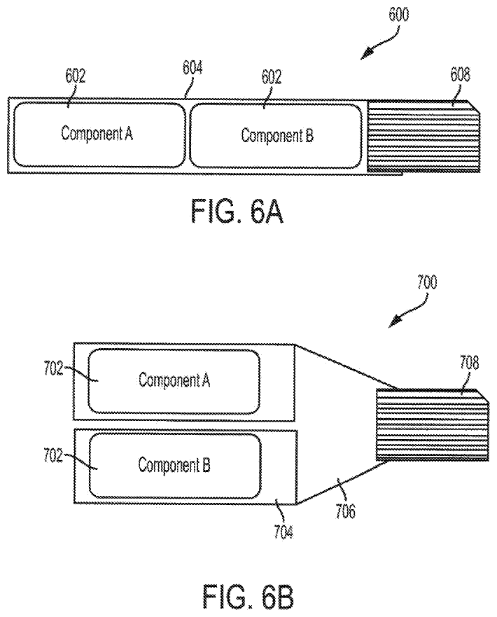

Tandem (top) and parallel (bottom) pop ampoule, dose applicators 600, 700 are depicted in FIG. 6A and FIG. 6B, respectively. As shown in FIG. 6A, ampoules 602 can be arranged in series with mixing taking place in a mixing tube 604. Alternatively, as shown in FIG. 6B, ampoules 702 can be arranged in parallel with mixing taking place in a mixing chamber space 706 defined between the ampoules 702 and a brush tip 708 mounted on the mixing tube 704. Alternately, the tip 708 can be a sponge or other arrangement so that the mixing can take place within the sponge itself.

Further, once the ampoules are broken, mixing can take place in the mixing tube and/or in a mixing chamber space between the ampoules and brush tip. Alternately, the mixing can take place within the sponge or brush tip itself. Metal oxide and polymer precursors and active agents like silver compounds mixed with solvents such as alcohols, such as isopropanol, ethanol, methanol, butanol, and alkanes such as, heptanes, xylene, hexanes and saturated cyclic hydrocarbons and their mixtures are placed together or separately in Ampoules. Crushing of the ampoules releases the contents and allows the components to mix and flow out to the applicator tip 608, 708. Once the mixed components are in the applicator tip, the coating is then brushed or dabbed onto the desired surface.

In one example, Component B contains titanium isopropoxide and methoxy functionalized polydimethylsiloxane at ratio of about 95:5, which is then diluted to about a 1.25% solution with isopropanol or a mixture of alcohol, (e.g., monohydric alcohol, polyhydric alcohol, unsaturated aliphatic alcohol, alicyclic alcohol and their mixtures as suitable solvents and carriers), isopropanol, and hexanes or heptanes. Component A is a therapeutic ingredient that mixes with and is transported by Component B, after the ampoules or chambers are opened.

Component A can be a silver compound mixed with solvents. Specifically, silver neodecanoate is used as a powder or preferably mixed with the solvent hexane or a mixture of hexane and xylene. In this embodiment, Component A is a silver compound mixed with solvents. Specifically, silver neodecanoate is used as a powder or preferably mixed with the solvent hexane, heptane or a mixture of hexane or heptane and xylene. A moderate dose of silver would consist of Component B chemicals mixed together at room temperature and by volume in the ratios of 1:0.01:82.8:6.54:2.0 (respectively for titanium isopropoxide, meth-oxy amine functionalized polydimethylsiloxane, and isopropanol). Component A chemicals mixed together at room temperature and by volume in the ratios of 6.54:2.0 (respectively for hexanes and the dopant 25% silver neodecanoate in xylenes). Each component would be placed in a glass ampoule 602, 702 or a sealed chamber within the dose applicator 600, 700. The color of a coating formed by this chemistry is generally white, clear to dark brown on a man-made substrate, skin or similar tissues.

Another example containing more silicone would be Component B chemicals mixed together at room temperature and by volume in the ratios of 1:0.1:82.8 (respectively for titanium isopropoxide, methoxy amine functionalized polydimethylsiloxane, and isopropanol). Component A chemicals mixed together at room temperature and by volume in the ratios of 6.54:2.0 (respectively for hexanes or heptanes and the dopant 25% silver neodecanoate in xylenes). Each component would be placed in a glass ampoule or a sealed chamber within the applicator. The color of a coating formed by this chemistry is generally white, clear to dark brown on a man-made substrate, skin or similar tissues.

Typically, metal oxide and polymer precursors and active agents like silver compounds mixed with solvents such as isopropanol, hexanes, heptanes in ampoules. Crushing of the ampoules by squeezing or external pressure releases the contents and allows the Components to mix and flow out to the applicator tip. The coating is then brushed or dabbed onto the desired surface. In this example, Component B contains titanium isopropoxide and meth-oxy functionalized polydimethylsiloxane at ratio of about 95:5, which is then diluted to about a 1.25% solution with isopropanol or a mixture of isopropanol and hexanes or heptanes. Component A is a therapeutic ingredient that mixes with and is transported by Component B, after the ampoules or chambers opened. In this example, Component A is a silver compound mixed with solvents. Specifically, silver neodecanoate is used as a powder or preferably mixed with the solvent hexane or heptane, or a mixture of xylene and hexane or heptane.

Preferably, each component is placed in a glass or plastic ampoule or a sealed chamber within the dose applicator. The color of a coating formed by this chemistry is generally white, clear to dark brown on a man-made substrate, skin or similar tissues. Another embodiment containing more silicone would be Component B chemicals mixed together at room temperature and by volume in the ratios of 1:0.1:82.8 (respectively for titanium isopropoxide, methoxy amine functionalized polydimethylsiloxane, and isopropanol). Component A chemicals mixed together at room temperature and by volume in the ratios of 6.54:2.0 (respectively for hexanes or heptanes and the dopant 25% silver neodecanoate in xylenes). Each component would be placed in a glass ampoule or a sealed chamber within the applicator. The color of a coating formed by this chemistry is generally white, clear to dark brown on a man-made substrate, skin or similar tissues.

Alternatively, Component A and Component B are dispensed with as an aerosol using pump action or propellant. A suitable double-chamber aerosol container for the packaging of products with several components which are to come into contact and be mixed at the time of use is described in U.S. Pat. Nos. 4,593,836 and 7,789,278.

One of the advantages of the pop ampule dose applicators and the aerosols described is that the coatings can be mixed and applied in a portable fashion at the point of use. Sterile applicators and aerosols described above would be used in the medical setting or surgical field to create the coatings directly to the skin as a surgical preparation or sunblock, an antimicrobial barrier on body tissues, or onto medical devices, instruments, hospital fixtures or medical implants. Examples of medical devices include orthopedic implants, fracture fixation, devices, plates, screws, rods, the surface of artificial joints, dental implants, medical tubing, catheters, urinary catheters, and wound dressings. Multiple applicators or aerosols can be used to apply multiple coatings as depicted in FIG. 2 and described in detail above.

### Active Ingredients for the Antifungal and Antimicrobial Drug Delivery System

In one embodiment, the active ingredient of the antifungal drug delivery system consists of at least one of the following seven antifungal agents within the specified concentration established for each ingredient:
(a) Clioquinol—up to 3 percent;
(b) Haloprogin—up to 1 percent;
(c) Miconazole nitrate—up to 2 percent;
(d) Povidone-iodine—up to 10 percent;
(e) Tolnaftate—up to 1 percent;
(f) Undecylenic acid, calcium undecylenate, copper undecylenate, and zinc undecylenate may be used individually or in any ratio that provides a total undecylenate concentration of 10 to 25 percent; and
(g) Clotrimazole—up to 1 percent.

In another embodiment, the active ingredient of the antimicrobial drug delivery system consists of at least one of the following six ointment/cream combinations within the specified concentration established for each ingredient:
(a) Bacitracin ointment containing, in each gram, up to 500 units of bacitracin in a suitable ointment base;
(b) Bacitracin zinc ointment containing, in each gram, up to 500 units of bacitracin zinc in a suitable ointment base;
(c) Chlortetracycline hydrochloride ointment containing, in each gram, up to 30 milligrams of chlortetracycline hydrochloride in a suitable ointment base;
(d) Neomycin sulfate ointment containing, in each gram, up to 3.5 milligrams of neomycin in a suitable water soluble or oleaginous ointment base;
(e) Neomycin sulfate cream containing, in each gram, up to 3.5 milligrams of neomycin in a suitable cream base; and
(f) Tetracycline hydrochloride ointment containing, in each gram, up to 30 milligrams of tetracycline hydrochloride in a suitable ointment base.

In yet another embodiment, the antimicrobial drug delivery system consists of at least one of the following 12 antimicrobial combinations of antibiotic active ingredients:
(1) Bacitracin-neomycin sulfate ointment containing, in each gram, up to 500 units of bacitracin and up to 3.5 milligrams of neomycin in a suitable ointment base;
(2) Bacitracin-neomycin sulfate-polymyxin B sulfate ointment containing, in each gram, in a suitable ointment base the following:
   (i) up to 500 units of bacitracin, up to 3.5 milligrams of neomycin, and up to 5,000 units of polymyxin B; or
   (ii) up to 400 units of bacitracin, up to 3.5 milligrams of neomycin, and up to 5,000 units of polymyxin B;
(3) Bacitracin-polymyxin B sulfate topical aerosol containing, in each gram, up to 500 units of bacitracin and up to 5,000 units of polymyxin B in a suitable vehicle, packaged in a pressurized container with suitable inert gases;
(4) Bacitracin zinc-neomycin sulfate ointment containing, in each gram, up to 500 units of bacitracin and up to 3.5 milligrams of neomycin in a suitable ointment base;
(5) Bacitracin zinc-neomycin sulfate-polymyxin B sulfate ointment containing, in each gram, in a suitable ointment base the following:
   (i) up to 400 units of bacitracin, up to 3 milligrams of neomycin, and up to 8,000 units of polymyxin B; or
   (ii) up to 400 units of bacitracin, up to 3.5 milligrams of neomycin, and up to 5,000 units of polymyxin B; or
   (iii) up to 500 units of bacitracin, up to 3.5 milligrams of neomycin, and up to 5,000 units of polymyxin B; or
   (iv) up to 500 units of bacitracin, up to 3.5 milligrams of neomycin, and up to 10,000 units of polymyxin B;
(6) Bacitracin zinc-polymyxin B sulfate ointment containing, in each gram, up to 500 units of bacitracin and up to 10,000 units of polymyxin B in a suitable ointment base;
(7) Bacitracin zinc-polymyxin B sulfate topical aerosol containing, in each gram, up to 120 units of bacitracin and up to 2,350 units of polymyxin B in a suitable vehicle, packaged in a pressurized container with suitable inert gases;
(8) Bacitracin zinc-polymyxin B sulfate topical powder containing, in each gram, up to 500 units of bacitracin and up to 10,000 units of polymyxin B in a suitable base;
(9) Neomycin sulfate-polymyxin B sulfate ointment containing, in each gram, up to 3.5 milligrams of neomycin and up to 5,000 units of polymyxin B in a suitable water miscible base;
(10) Neomycin sulfate-polymyxin B sulfate cream containing, in each gram, up to 3.5 milligrams of neomycin and up to 10,000 units of polymyxin B in a suitable vehicle;
(11) Oxytetracycline hydrochloride-polymyxin B sulfate ointment containing, in each gram, up to 30 milligrams of oxytetracycline and up to 10,000 units of polymyxin B in a suitable ointment base; and
(12) Oxytetracycline hydrochloride-polymyxin B sulfate topical powder containing, in each gram, up to 30 milligrams of oxytetracycline and up to 10,000 units of polymyxin B with a suitable filler.

In another alternative embodiment, the drug delivery system consists of at least one of the following six combinations of first aid antibiotic active ingredients and local anesthetic active ingredients:
(1) Bacitracin ointment containing, in each gram, up to 500 units of bacitracin and any single generally recognized as safe and effective amine or “caine”-type local anesthetic active ingredient in a suitable ointment base;
(2) Bacitracin-neomycin sulfate-polymyxin B sulfate ointment containing, in each gram, in a suitable ointment base the following:
   (i) up to 500 units of bacitracin, up to 3.5 milligrams of neomycin, up to 5,000 units of polymyxin B, and any single generally recognized as safe and effective amine or “caine”-type local anesthetic active ingredient; or
   (ii) up to 400 units of bacitracin, up to 3.5 milligrams of neomycin, up to 5,000 units of polymyxin B, and any single generally recognized as safe and effective amine or “caine”-type local anesthetic active ingredient;
(3) Bacitracin-polymyxin B sulfate topical aerosol containing, in each gram, up to 500 units of bacitracin and up to 5,000 units of polymyxin B and any single generally recognized as safe and effective amine or “caine”-type local anesthetic active ingredient in a suitable vehicle, packaged in a pressurized container with suitable inert gases;
(4) Bacitracin zinc-neomycin sulfate-polymyxin B sulfate ointment containing, in each gram, in a suitable ointment base the following:
   (i) up to 400 units of bacitracin, up to 3 milligrams of neomycin, up to 8,000 units of polymyxin B, and any single generally recognized as safe and effective amine or “caine”-type local anesthetic active ingredient; or
   (ii) up to 400 units of bacitracin, up to 3.5 milligrams of neomycin, up to 5,000 units of polymyxin B, and any single generally recognized as safe and effective amine or “caine”-type local anesthetic active ingredient; or
   (iii) up to 500 units of bacitracin, up to 3.5 milligrams of neomycin, up to 5,000 units of polymyxin B, and any single generally recognized as safe and effective amine or “caine”-type local anesthetic active ingredient; or
   (iv) up to 500 units of bacitracin, up to 3.5 milligrams of neomycin, up to 10,000 units of polymyxin B, and any single generally recognized as safe and effective amine or “caine”-type local anesthetic active ingredient;
(5) Bacitracin zinc-polymyxin B sulfate ointment containing, in each gram, up to 500 units of bacitracin, up to 10,000 units of polymyxin B, and any single generally recognized as safe and effective amine or “caine”-type local anesthetic active ingredient in a suitable ointment base; and
(6) Neomycin sulfate-polymyxin B sulfate cream containing, in each gram, up to 3.5 milligrams of neomycin, up to 10,000 units of polymyxin B, and any single generally recognized as safe and effective amine or “caine”-type local anesthetic active ingredient in a suitable vehicle.”

In yet another alternative embodiment, the active ingredient of the antimicrobial drug delivery system consists of at least one of the following six antifungal drugs within the specified concentration established for each ingredient:

(a) Benzoyl peroxide, 2.5 to 10 percent;

(b) resorcinol, up to 2 percent, when combined with sulfur;

(c) resorcinol monoacetate, up to 3 percent, when combined with sulfur;

(d) Salicylic acid, 0.5 to 2 percent;

(e) Sulfur, 3 to 10 percent; and

Sulfur, 3 to 8 percent, when combined with resorcinol or resorcinol monoacetate.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention, and are not intended to limit the invention.

### Example 1 Coating

Coatings are formed on an article, medical device or implant, such as a catheter, fracture fixation device, joint replacement, wound dressing or applied directly to a structure or tissues of the body, such as the skin, bone, muscle or organs from a composition consisting of a liquid precursor solution of titanium isopropoxide, methoxy amine functionalized polydimethylsiloxane, isopropanol and non-polar solvents such as hexanes and heptanes and the dopant silver neodecanoate.

For the reservoir layer, these chemicals are mixed together at room temperature and by volume in the ratios of 1:0.01:10.35:0.817:2.0 (for titanium isopropoxide, methoxy amine functionalized polydimethylsiloxane, isopropanol and non-polar solvents such as hexanes and heptanes and the dopant silver neodecanoate respectively), while the outermost layer forming the diffusion barrier is mixed in the ratios of 1:0.1:10.35:0.817:0.2 (respectively). In this case, the reservoir layer is made from a solution containing ten times more silver than the barrier layer. The reservoir chemistry also contains ten times more PDMS, to aid in the release of the silver ions.

The barrier layer has a lower concentration of PDMS to improve mammalian cellular adhesion in this instance. The reservoir layer is sprayed onto the substrate using the chemistry described for this layer using a gravity fed sprayer with an air pressure of 40 psi to deposit the solvents and precursors uniformly onto the cleaned surface of the medical device substrate. The solvent is allowed to flash in at room temperature and under atmospheric conditions, leaving a yellow to white coating of approximately 100-500 nm in thickness. Thinner coatings may alternately be applied to create a spectrum of interference colored appearance from red to purple and blue.

A single or multiple layers of the reservoir composition may be applied by the same technique until the desired volume of dopants is applied to the device. A slightly elevated temperature of approximately 37° C. to 41° C. or up to 80° C. could be used to speed flashing and evaporation of the solvents. This elevated temperature may be applied by resistance heating, convection, infrared or ultraviolet lighting systems.

A final layer of coating is then applied to form the diffusion barrier layer using the second chemical composition and a similar spraying technique and allowed to air-dry, forming a final layer of approximately 100-500 nm in thickness.

Alternately, the coatings can be applied using electrostatic spraying techniques, airless spraying or sprayed using inert or active gases or hydrocarbons as the propellant in place of air.

### Example 2 Dip Coating

A dip coating method may be used to apply the coating system. In this case, the volume of the solvents would be increased by approximately eight fold to facilitate the formation of a unified coating.

For this method, the reservoir layer chemicals would be mixed together at room temperature and by volume in the ratios of 1:0.01:82.8:6.54:2.0 (respectively for titanium isopropoxide, methoxy amine functionalized polydimethylsiloxane, isopropanol and non-polar solvents such as hexanes and heptanes and the dopant silver neodecanoate), while the outermost layer forming the diffusion barrier is mixed in the ratios of 1:0.1:82.8:6.54:0.2, respectively.

The medical device would then be dipped into the reservoir solution at a rate of approximately 4 inches per second and withdrawn at a rate of approximately 2 inches per second, under normal atmospheric conditions and room temperature. A slightly elevated temperature of approximately 37° C. to 41° C. or up to 80° C. can be used to speed flashing and evaporation of the solvents. This elevated temperature may be applied by resistance heating, convection, infrared or ultraviolet lighting systems. The resulting coating being yellow to white and approximately 100-500 nm in thickness. The dipping may be repeated to achieve the desired thickness and loading of active agents.

A final layer of dip coating is then applied to form the diffusion barrier layer using the second chemical composition and a similar dipping technique and allowed to air-dry with or without the addition of heat and photon energy, forming a final yellow to white layer of approximately 100-500 nm in thickness. Alternatively, thinner coatings may be applied to create a spectrum of interference colored appearance from red to purple and blue. The color of the final coating was directly related to the thickness of the coatings as is achieved with anodization of titanium alloys. The invention includes the use of these chemistries and methods to create these colors on devices.

### Example 3 Coatings with Broad Spectrum Photoactivity

Solutions formed from solid state suspensions for metal oxides and polymers can be used to create coatings with broad spectrum photoactivity as described in the several patents and applications.¹⁷ This photoactivity is related to the formation of valence electrons in the presence of photons from x-rays to infrared which also produces superoxide in the presence of moisture or water.

The reaction does not yield a large amount of hydroxyl radials when compared to the photocatalytic properties of crystalline titanium oxide exposed to ultraviolet radiation. The addition or formation of iron particles within the coating promotes increased hydroxyl radical production from superoxide using the Fenton reaction. This could be added in the form of iron oxide nano particles or by use of iron (II) sulfate or similar iron source for the Fenton reaction. The production of the hydroxyl radical by this reaction is effective for decontamination of surrounding area and destruction of microbes such as bacteria, viruses and fungi on medical devices, until the iron eluted from the coating and during irradiation by a broad spectrum of radiation, such as medical x-rays or phototherapy using ultraviolet, visible and infrared wavelengths. Coating systems and irradiation schemes, as disclosed herein, may also be used to create general purpose filters and purification systems to destroy organic compounds and microbes in air and liquid purification systems.

For the reservoir layer, these chemicals are mixed together at room temperature and by volume in the ratios of 1:0.01:10.35:0.817:2.0 (for titanium isopropoxide to methoxy amine functionalized polydimethylsiloxane to isopropanol to non-polar solvents such as hexanes and heptanes to the dopant silver neodecanoate, respectively), while the outermost layer forming the diffusion barrier was mixed in the ratios of 1:0.02:10.35:0.817:0.2:0.1 (for titanium isopropoxide to methoxy amine functionalized polydimethylsiloxane to isopropanol to non-polar solvents such as hexanes and heptanes to the dopant silver neodecanoate and a 20% solution (wt/vol) in isopropanol of ferric nitrate nonahydrate or ferric chloride hexahydrate or ferric chloride salt (iron compounds per Gash et al.¹⁸) or nano (˜10 nm) iron oxide (II, III) particulate, respectively). These two types of layers were applied by spray coating as described in Example 1 or diluted with eight times more solvent and dip coated according to Example 2. These two coating may be applied to the inside of a 20 mL glass scintillation vessel to create a photocatalytic reaction chamber for producing hydroxyl and superoxide in the presence of broad spectrum photon irradiation from x-rays to infrared.

### Example 4 Coatings with Extended Spectral Response

Alternatively, water, acids or bases may be mixed with the coating solutions to form sols and particulate forms within the solvent carriers. Ceramics, plastics, metals, oxides or salts may be mixed in the coating solutions to influence the bioactivity mechanical, physical properties or controlled delivery properties of the coatings. Crystalline titanium oxide particles within sols or nano particles (i.e., AEROXIDE® TiO2 P 25 available from the Evonik Degussa Corporation) may be mixed in with the solutions to impart photocatalytic properties to the coatings under ultraviolet radiation. This can be used to enhance the generation of electron and hole pairs in the presence of photons, which produce reactive oxygen species, namely the hydroxyl radical and superoxide in the presence of moisture or water. This enables the conversion of the non-discrete valance electrons and holes created during photon irradiation of the amorphous titanium oxide and polymer composition coatings into discrete electrons and holes associated with crystalline titanium oxide and the photocatalytic process produced by the crystalline semi-conductor material. This allows delivery of discrete electrical charges from broad spectrum photon irradiation, thus extending the phenomena seen with the dye-sensitized titanium oxide solar cell, below the ultraviolet range into x-rays and above the visible range into the infrared spectrum. Extending the spectral response is an advantage over the prior art.¹⁹

For the reservoir layer, these chemicals are mixed together at room temperature and by volume in the ratios of 1:0.01:10.35:0.817:2.0 (for titanium isopropoxide to methoxy amine functionalized polydimethylsiloxane to isopropanol to non-polar solvents such as hexanes and heptanes to the dopant silver neodecanoate, respectively), while the outermost layer forming the diffusion barrier is mixed in the ratios of 1:0.02:10.35:0.817:1 (for titanium isopropoxide to methoxy amine functionalized polydimethylsiloxane to isopropanol to non-polar solvents such as hexanes and heptanes to 20% Degusa P25 nano crystalline titanium oxide solution in isopropanol (wt to volume)). These two types of layers may be applied by spray coating as described in Example 1 or diluted with eight times more solvent and dip coated according to Example 2. These two coating may be applied to the inside of a 20 mL glass scintillation vessel to create a photocatalytic reaction chamber for producing hydroxyl and superoxide in the presence of broad spectrum photon irradiation from x-rays to infrared.

### Example 5 Outer Barrier Layer Incorporating Elution and/or Immobilization Drugs

A drug may be incorporated into the outer barrier layer for elution and/or immobilization. For the reservoir layer, these chemicals are mixed together at room temperature and by volume in the ratios of 1:10 (25% silver neodecanoate in xylenes to isopropanol respectively), while the outermost layer forming the diffusion barrier is mixed in the ratios of 1:0.03:10.35:0.817:0.002:1 (for titanium isopropoxide to methoxy amine functionalized polydimethylsiloxane to isopropanol to non-polar solvents such as hexanes and heptanes to 25% silver neodecanoate in xylenes to 10% solution/slurry of Lortadine or diphenhydramine in ethanol or isopropanol (wt/vol)). These two types of layers are applied by spray coating as described in Example 1 or diluted with eight times more solvent and dip coated according to Example 2.

### Example 6 Drug Delivery System and Applicator

Drugs may be dissolved within the carrier for the coating system or dissolved within the metal oxide precursor and or polymer precursor. Ibuprofen is an example of a drug which is soluble within the alcohol-based carrier system used for applying the coatings. In this case, 0.2 grams of ibuprofen are dissolved in 1 mL of isopropanol to create a saturated or near saturated solution. The isopropanol with dissolved drug is then used in place of isopropanol to create one or more of the layers described in Examples 1-5. The anti-inflammatory drugs phenylbutazone and nabumetone, may be dissolved into the polydimethylsiloxane at a concentration of 80 mg/L and the polydimethylsiloxane with dissolved drug used to replace the polydimethylsiloxane described in Examples 1-5. The 25% silver neodecanoate in xylenes are mixed with drugs soluble in decanoaic acid, such as nandrolone, fluphenazine, bromperidol, haloperidol and vanoxerine and the silver necodecanoate with dissolved drug use to replace the silver neodecanoate in 25% xylenes described in Examples 1-5.

One or both layers of the delivery system are applied using a pen-type applicator, directly to the substrate which may be skin other tissues or medical devices. The pen consists of a barrel with one or more compartments for holding the procurer solutions separate until the time of application and an absorbent tip for dispensing a thin film of the precursor solutions.

### Example 7 Antifungal and Antimicrobial Drug Delivery Systems and Applicators

In one example, the active ingredient of the antifungal drug delivery system consists of any one of the following seven active ingredients within the specified concentration established for each ingredient:
(a) Clioquinol—3 percent;
(b) Haloprogin—1 percent;
(c) Miconazole nitrate—2 percent;
(d) Povidone-iodine—10 percent;
(e) Tolnaftate—1 percent;
(f) Undecylenic acid, calcium undecylenate, copper undecylenate, and zinc undecylenate may be used individually or in any ratio that provides a total undecylenate concentration of 10 to 25 percent; or
(g) Clotrimazole—1 percent.

In another example, the active ingredient of the antimicrobial drug delivery system consists of any one of the following six ointments/cream within the specified concentration established for each ingredient:
(a) Bacitracin ointment containing, in each gram, 500 units of bacitracin in a suitable ointment base;
(b) Bacitracin zinc ointment containing, in each gram, 500 units of bacitracin zinc in a suitable ointment base;
(c) Chlortetracycline hydrochloride ointment containing, in each gram, 30 milligrams of chlortetracycline hydrochloride in a suitable ointment base;
(d) Neomycin sulfate ointment containing, in each gram, 3.5 milligrams of neomycin in a suitable water soluble or oleaginous ointment base;
(e) Neomycin sulfate cream containing, in each gram, 3.5 milligrams of neomycin in a suitable cream base; or
(f) Tetracycline hydrochloride ointment containing, in each gram, 30 milligrams of tetracycline hydrochloride in a suitable ointment base.

In yet another example, the antimicrobial drug delivery system consists of any one of the following 12 combinations of antimicrobial active ingredients:
(1) Bacitracin-neomycin sulfate ointment containing, in each gram, 500 units of bacitracin and 3.5 milligrams of neomycin in a suitable ointment base;
(2) Bacitracin-neomycin sulfate-polymyxin B sulfate ointment containing, in each gram, in a suitable ointment base the following:
   (i) 500 units of bacitracin, 3.5 milligrams of neomycin, and 5,000 units of polymyxin B; or
   (ii) 400 units of bacitracin, 3.5 milligrams of neomycin, and 5,000 units of polymyxin B;
(3) Bacitracin-polymyxin B sulfate topical aerosol containing, in each gram, 500 units of bacitracin and 5,000 units of polymyxin B in a suitable vehicle, packaged in a pressurized container with suitable inert gases;
(4) Bacitracin zinc-neomycin sulfate ointment containing, in each gram, 500 units of bacitracin and 3.5 milligrams of neomycin in a suitable ointment base;
(5) Bacitracin zinc-neomycin sulfate-polymyxin B sulfate ointment containing, in each gram, in a suitable ointment base the following:
   (i) 400 units of bacitracin, 3 milligrams of neomycin, and 8,000 units of polymyxin B; or
   (ii) 400 units of bacitracin, 3.5 milligrams of neomycin, and 5,000 units of polymyxin B; or
   (iii) 500 units of bacitracin, 3.5 milligrams of neomycin, and 5,000 units of polymyxin B; or
   (iv) 500 units of bacitracin, 3.5 milligrams of neomycin, and 10,000 units of polymyxin B;
(6) Bacitracin zinc-polymyxin B sulfate ointment containing, in each gram, 500 units of bacitracin and 10,000 units of polymyxin B in a suitable ointment base;
(7) Bacitracin zinc-polymyxin B sulfate topical aerosol containing, in each gram, 120 units of bacitracin and 2,350 units of polymyxin B in a suitable vehicle, packaged in a pressurized container with suitable inert gases;
(8) Bacitracin zinc-polymyxin B sulfate topical powder containing, in each gram, 500 units of bacitracin and 10,000 units of polymyxin B in a suitable base;
(9) Neomycin sulfate-polymyxin B sulfate ointment containing, in each gram, 3.5 milligrams of neomycin and 5,000 units of polymyxin B in a suitable water miscible base;
(10) Neomycin sulfate-polymyxin B sulfate cream containing, in each gram, 3.5 milligrams of neomycin and 10,000 units of polymyxin B in a suitable vehicle;
(11) Oxytetracycline hydrochloride-polymyxin B sulfate ointment containing, in each gram, 30 milligrams of oxytetracycline and 10,000 units of polymyxin B in a suitable ointment base; or
(12) Oxytetracycline hydrochloride-polymyxin B sulfate topical powder containing, in each gram, 30 milligrams of oxytetracycline and 10,000 units of polymyxin B with a suitable filler.

In another alternative embodiment, the drug delivery system consists of any one of the following six combinations of first aid antibiotic active ingredients and local anesthetic active ingredients:
(1) Bacitracin ointment containing, in each gram, 500 units of bacitracin and any single generally recognized as safe and effective amine or “caine”-type local anesthetic active ingredient in a suitable ointment base;
(2) Bacitracin-neomycin sulfate-polymyxin B sulfate ointment containing, in each gram, in a suitable ointment base the following:
   (i) 500 units of bacitracin, 3.5 milligrams of neomycin, 5,000 units of polymyxin B, and any single generally recognized as safe and effective amine or “caine”-type local anesthetic active ingredient; or
   (ii) 400 units of bacitracin, 3.5 milligrams of neomycin, 5,000 units of polymyxin B, and any single generally recognized as safe and effective amine or “caine”-type local anesthetic active ingredient;
(3) Bacitracin-polymyxin B sulfate topical aerosol containing, in each gram, 500 units of bacitracin and 5,000 units of polymyxin B and any single generally recognized as safe and effective amine or “caine”-type local anesthetic active ingredient in a suitable vehicle, packaged in a pressurized container with suitable inert gases;
(4) Bacitracin zinc-neomycin sulfate-polymyxin B sulfate ointment containing, in each gram, in a suitable ointment base the following:
   (i) 400 units of bacitracin, 3 milligrams of neomycin, 8,000 units of polymyxin B, and any single generally recognized as safe and effective amine or “caine”-type local anesthetic active ingredient; or
   (ii) 400 units of bacitracin, 3.5 milligrams of neomycin, 5,000 units of polymyxin B, and any single generally recognized as safe and effective amine or “caine”-type local anesthetic active ingredient; or
   (iii) 500 units of bacitracin, 3.5 milligrams of neomycin, 5,000 units of polymyxin B, and any single generally recognized as safe and effective amine or “caine”-type local anesthetic active ingredient; or
   (iv) 500 units of bacitracin, 3.5 milligrams of neomycin, 10,000 units of polymyxin B, and any single generally recognized as safe and effective amine or “caine”-type local anesthetic active ingredient;
(5) Bacitracin zinc-polymyxin B sulfate ointment containing, in each gram, 500 units of bacitracin, 10,000 units of polymyxin B, and any single generally recognized as safe and effective amine or “caine”-type local anesthetic active ingredient in a suitable ointment base; or
(6) Neomycin sulfate-polymyxin B sulfate cream containing, in each gram, 3.5 milligrams of neomycin, 10,000 units of polymyxin B, and any single generally recognized as safe and effective amine or “caine”-type local anesthetic active ingredient in a suitable vehicle.”

In yet another alternative example, the active ingredient of the antimicrobial drug delivery system consists of any one of the following six active ingredients within the specified concentration established for each ingredient:

(a) Benzoyl peroxide, 2.5 to 10 percent;

(b) resorcinol, 2 percent, when combined with sulfur;

(c) resorcinol monoacetate, 3 percent, when combined with sulfur;

(d) Salicylic acid, 0.5 to 2 percent;

(e) Sulfur, 3 to 10 percent; or

(f) Sulfur, 3 to 8 percent, when combined with resorcinol or resorcinol monoacetate.

A tandem pop ampoule, dose applicator is depicted in FIG. 6A is used. Metal oxide and polymer precursors and active agents like silver compounds mixed with solvents such as isopropanol, hexane or heptane are placed together or separately in Ampoules. Crushing of the ampoules releases the contents and allows the Component A and Component B to mix and flow out to the applicator tip. The coating is then brushed or dabbed onto the desired surface. In this example, Component B contains titanium isopropoxide and methoxy functionalized polydimethylsiloxane at ratio of about 95:5, which is then diluted to about a 1.25% solution with isopropanol or a mixture of isopropanol and hexane or heptane. Component A is a therapeutic ingredient that mixes with and is transported by Component B, after the ampoules or chambers opened. In this example, Component A is a silver compound mixed with solvents. Specifically, silver neodecanoate is used as a powder or preferably mixed with the solvent hexane or a mixture of hexane and xylene.

A moderate dose of silver would consist of Component B chemicals mixed together at room temperature and by volume in the ratios of 1:0.01:82.8:6.54:2.0 (respectively for titanium isopropoxide, methoxy amine functionalized polydimethylsiloxane, and isopropanol). Component A chemicals mixed together at room temperature and by volume in the ratios of 6.54:2.0 (respectively for hexane or heptane and the dopant 25% silver neodecanoate in xylenes). Each component would be placed in a glass ampoule or a sealed chamber within the dose applicator. The color of a coating formed by this chemistry is generally white, clear to dark brown on a man-made substrate, skin or similar tissues.

Another example containing more silicone would be Component B chemicals mixed together at room temperature and by volume in the ratios of 1:0.1:82.8 (respectively for titanium isopropoxide, methoxy amine functionalized polydimethylsiloxane, and isopropanol). Component A chemicals mixed together at room temperature and by volume in the ratios of 6.54:2.0 (respectively for hexane or heptane and the dopant 25% silver neodecanoate in xylenes). Each component would be placed in a glass ampoule or a sealed chamber within the applicator. The color of a coating formed by this chemistry is generally white, clear to dark brown on a manmade substrate, skin or similar tissues.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### REFERENCES

¹U.S. Pat. No. 7,906,132, Anti-infectious, biocompatible titanium coating for implants, and method for the production thereof issued to Ziegler et al. on Mar. 15, 2011.
² Jarrell J D, Dolly B, Morgan J R. Controlled release of vanadium from titanium oxide coatings for improved integration of soft tissue implants. J Biomed Mater Res A, Volume 90A, Issue 1, Pages 272-281, June 2009.
³ Jarrell J D, Dolly B, Morgan J R. Rapid screening, in vitro study of metal oxide and polymer hybrids as delivery coatings for improved soft-tissue integration of implants. J Biomed Mater Res A, Volume 92A, Issue 3, Pages 10941104, 1 Mar. 2010.
⁴ The Merck Manual of Diagnosis and Therapy, 19th Edition, published by Merck Sharp & Dohme Corp., 2011 (ISBN 978-0-911910-19-3).
⁵ The Encyclopedia of Molecular Cell Biology and Molecular Medicine, Robert S. Porter et al. (eds.), published by Blackwell Science Ltd., 1999-2012 (ISBN 9783527600908).
⁶ Molecular Biology and Biotechnology: a Comprehensive Desk Reference, Robert A. Meyers (ed.), published by VCH Publishers, Inc., 1995 (ISBN 1-56081-569-8).
⁷ Immunology by Werner Luttmann, published by Elsevier, 2006.
⁸ Janeway's Immunobiology, Kenneth Murphy, Allan Mowat, Casey Weaver (eds.), Taylor & Francis Limited, 2014 (ISBN 0815345305, 9780815345305).
⁹ Lewin's Genes XI, published by Jones & Bartlett Publishers, 2014 (ISBN-1449659055).
¹⁰ Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414).
¹¹ Davis et al., Basic Methods in Molecular Biology, Elsevier Science Publishing, Inc., New York, USA (2012) (ISBN 044460149X).
¹² Laboratory Methods in Enzymology: DNA, Jon Lorsch (ed.) Elsevier, 2013 (ISBN 0124199542).
¹³ Current Protocols in Molecular Biology (CPMB), Frederick M. Ausubel (ed.), John Wiley and Sons, 2014 (ISBN 047150338X, 9780471503385).
¹⁴ Current Protocols in Protein Science (CPPS), John E. Coligan (ed.), John Wiley and Sons, Inc., 2005.
¹⁵ Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737).
¹⁶ See, e.g., Isselbacher et al. (1996) Harrison's Principles of Internal Medicine, 13 ed., 1814-1882.
¹⁷ U.S. Pat. No. 8,080,223 issued Dec. 20, 2011 to Jarrell et al. entitled: “Method of Making a Composite from Metal Oxide and Polymer Precursors”; U.S. Pat. No. 7,868,078 issued Jan. 11, 2011 to Jarrell entitled: “Composition Including Metal Oxide and Polymer”; U.S. Published Application No. 2009/0104473, U.S. Ser. No. 12/253,354 filed Oct. 17, 2008 entitled: “Novel Compositions and Related Methods, Coatings, and Articles”; and U.S. Published Application No. 2011/0092870, U.S. Ser. No. 12/975,218 filed Dec. 21, 2010 entitled Article Comprising of an Object with Coating from Polymer and Metal Oxide Precursor.”
¹⁸ Gash, et. al., Use of epoxides in the sol-gel synthesis of porous iron (III) Oxide Monoliths from Fe(III) Salts. Chem. Mater. 2001, 13, 999-1007.
¹⁹ U.S. Pat. No. 5,084,365 to Gratzel et. al., entitled: “Photo-Electrochemical Cell and Process of Making Same, provides background information.”

1. An applicator for preparing a mixture at a point of use comprising:
a housing defining an interior and an opening;
first and second containers contained in the interior, wherein the first container contains a first component and the second container contains a second component; and
an applicator tip secured in the opening so that when the first and second components are mixed to form the mixture and the mixture can egress from the application tip; and
the component in the first container contains precursors which form a metal oxide and polymer hybridized matrix for controlled delivery of one or more antifungal agent; and
the component in the second container contains the one or more antifungal and/or antimicrobial agent.

2. The applicator as recited in claim 1, wherein the chemistry in the first container forms a matrix of at least one metal oxide consisting of titanium oxide or zirconium oxide or niobium oxide or tantalum oxide or a mixture of these oxides.

3. The applicator as recited in claim 1, wherein the chemistry in the second container contains at least one metal, metal oxide, metal salt or metal ester of silver, vanadium, zinc or copper or a mixture of these metals or oxides.

4. The applicator as recited in claim 1, wherein the metal or metal oxide from the second container becomes trapped within the matrix formed by the chemistry in the first container.

5. The applicator as recited in claim 1, where in the first container contains precursors which form a titanium oxide and siloxane matrix.

6. The applicator as recited in claim 1, where in the second container includes a silver salt, sliver ester or silver neodecanoate.

7. The applicator as recited in claim 1, further comprising a third container contained in the interior, wherein the third container contains a third component so that when the first, second and third containers are broken, the first, second and third components mix and egress from the application tip.

8. (canceled)

9. The applicator as recited in claim 1, wherein the mixture of the first and second components forms a coating that acts as a diffusion barrier, wherein the coating covers a reservoir coating.

10. (canceled)

11. An applicator for preparing and applying a barrier layer and drug reservoir layer at a point of use comprising:
a housing defining an interior and an opening;
first and second containers contained in the interior, wherein the first container contains a first component and the second container contains a second component; and
an applicator tip secured in the opening so that when the first and second components can egress from the application tip; and
the component in the first container contains precursors which form a barrier layer; and the component in the second forms an antifungal agent reservoir.

12. The applicator as recited in claim 11, further comprising a third container contained in the interior, wherein the third container contains a third component so that when broken the component egresses from the application tip.

13-21. (canceled)

22. An applicator for preparing a coating at a point of use comprising:
a housing defining an interior and an opening;
at least one container contained in the interior, wherein the container contains a first component; and
an applicator tip secured in the opening so that a component can egress from the application tip; and
at least one component contains precursors which form a metal oxide and polymer hybridized matrix on a surface.

23. The applicator as recited in claim 22, wherein the metal oxide and polymer hybridized matrix forms a coating on the tissues of the body, such as the skin, epithelial layers, bone, muscle or organs.

24. The applicator as recited in claim 22, wherein the metal oxide and polymer hybridized matrix contains an antifungal agent.

25. The applicator as recited in claim 22, wherein the precursors form at least one metal oxide consisting of titanium oxide or zirconium oxide or niobium oxide or tantalum oxide or a mixture of these oxides.

26. The applicator as recited in claim 22, wherein the precursors form at least one polymer consisting of polysiloxane or polydimethylsiloxane or functionalized polysiloxane or functionalized polydimethylsiloxane.

27. The applicator as recited in claim 22, wherein the metal oxide and polymer hybridized matrix includes a saturated fatty acid or medium-chain triglyceride such as decanoic acid.

28-32. (canceled)
